(19) United States
(12) Patent Application Publication
(10) Pub. No.: US 2006/0177914 A1
(43) Pub. Date: Aug. 10, 2006

(54) Coupled cofactor-dependent enzymatic reaction system

(76) Inventors: Claudia Rollmann, Alzenau (DE); Harald Groger, Hanau (DE); Werner Hummel, Titz (DE); Andreas Bomarius, Atlanta, GA (US); Karlheinz Drauz, Freigericht (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/546,733

(22) PCT Filed: Mar. 17, 2004
(86) PCT No.: PCT/EP04/02727

§ 371 Date: Aug. 24, 2005

(30) Foreign Application Priority Data
Mar. 27, 2003 (DE) 10313972.9

(51) Int. Cl. C12P 7/18; C12N 9/02

(52) U.S. Cl. 435/158; 435/189

(57) ABSTRACT

The present application relates to a reaction system in which chemically valuable compounds can be obtained in high enantiomer purities with the aid of a coupled enzymatically operating transformation process. The coupled enzymatically operating reaction system in this context comprises an enzymatic transformation reaction which proceeds with the consumption of a cofactor, and recycling of the cofactor consumed, which proceeds enzymatically, wherein the procedure is carried out in a homogeneous aqueous solvent system comprising an organic hydrocarbon having at least two hydroxyl or ether groups.

The present invention relates to a coupled enzymatically operating reaction system which is distinguished in that it is carried out in a homogeneous solvent mixture. In particular, the invention relates to a reaction system comprising a cofactor-dependent enzymatic transformation of an organic compound, wherein the cofactor is regenerated enzymatically in the same system.

The production of optically active organic compounds, e.g. alcohols and amino acids, by a biocatalytic route is increasingly gaining importance. The coupled use of two dehydrogenases with cofactor regeneration has emerged as a route for the large-scale industrial synthesis of these compounds (DE19753350).

Equation 1:

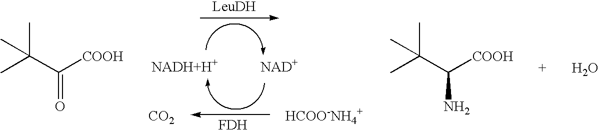

In situ regeneration of NADH with the NAD-dependent formate dehydrogenase in the reductive amination of trimethyl pyruvate to give L-tert-leucine (Bommarius et al. Tetrahedron Asymmetry 1995, 6, 2851-2888).

In addition to their catalytic property and efficiency, the biocatalysts efficiently employed in an aqueous medium furthermore have the advantage that in contrast to a large number of synthetic metal-containing catalysts, the use of metal-containing starting substances, in particular those which contain heavy metals and are therefore toxic, can be dispensed with. The use of expensive and furthermore hazardous reducing agents, such as, for example, borane, in the case of asymmetric reduction can also be dispensed with.

Nevertheless, difficulties occur in the reaction of substrates which are poorly water-soluble. Similar difficulties exist in the case of poorly water-soluble products. A solution which is conceivable in principle would be to carry out the biocatalytic reduction in a polar organic solvent or an aqueous solution thereof. In this case, both the enzymes and the substrate and, where appropriate, the product should be water-soluble. A general disadvantage of a direct presence of an organic solvent, however, is the considerable reduction which generally occurs in the enzyme activity under these conditions (see e.g. Anderson et al., Biotechnol. Bioeng. 1998, 57, 79-86). In particular, FDH as the only formate dehydrogenase employed hitherto on an industrial scale and accessible in commercial amounts unfortunately has a high sensitivity towards organic solvents. This also manifests itself in comparison example 1 using DMSO, sulfolane, MTBE, acetone, isopropanol and ethanol as the organic solvent component in added amounts of in each case 10% (see FIG. 1). Various set-ups are known to solve this problem of stabilization of the formate dehydrogenase from Candida boidinii in the presence of organic solvents, e.g. carrying out reactions by the additional use of surfactants as surface-active substances. Disadvantages here, however, are the rate of reaction, which is reduced by about a factor of 40 (!), and the inhibition of formate dehydrogenase which occurs (B. Orlich et al., Biotechnol. Bioeng. 1999, 65, 357-362.). The authors furthermore note that because of the low stability of the alcohol dehydrogenase employed, a reduction process under these conditions of a microemulsion is not economical.

A further possibility in principle for carrying out biocatalytic reactions consists of the use of immobilized enzymes in the organic solvent or the use of enzymes in a homogeneous solution comprising water and a water-miscible organic solvent. However, these techniques in which direct contact occurs between the organic solvent and enzyme are limited to a few enzyme classes, in particular hydrolases. It is thus noted in DE4436149 that the “direct presence of organic solvents (water-miscible or water-immiscible) is tolerated by only a few enzymes which belong to the class of hydrolases”. A few further examples from other enzyme classes have indeed since become known (thus, inter alia, oxynitrilases), but the statement made in DE4436149 is still applicable to the majority of enzymes. An efficient immobilization of the FDH from Candida boidinii is thus not known. Furthermore, the immobilization itself is associated with additional costs due to the immobilization step and the immobilization materials.

Industrially, processes have therefore been developed which avoid the presence of organic solvents because of the risk of deactivation or denaturing of the enzymes. DE4436149 thus describes a process in which the product is extracted from the reaction solution into an organic solvent through a membrane, in particular a hydrophobic membrane, which is permeable to the product. Compared with a standard process in a stirred tank reactor, however, this process requires significantly more technical outlay, especially since the organic membranes required are also an additional cost factor. Furthermore, this method is suitable only for continuous processes.

Summarizing, it can be said that thus no process which helps to bypass the abovementioned disadvantages is known.

The object of the present invention was therefore to provide a possibility such that, in particular, poorly water-soluble organic compounds can be rendered accessible to a coupled cofactor-dependent enzymatic reaction to an adequate extent such that the possibility can be used on an industrial scale under, in particular, economically and ecologically advantageous conditions.

This object is achieved according to the claims. Claims 1 to 8 relate to a reaction system which operates according to the invention. Claim 9 protects a device. Claim 10 relates to a process which operates according to the invention, while claims 11 and 12 relate to preferred uses of the reaction system according to the invention.

By providing a coupled enzymatic reaction system comprising a cofactor-dependent enzymatic transformation of an organic compound and an enzymatic regeneration of the cofactor, the reaction system operating in a homogeneous aqueous solvent system comprising an organic hydrocarbon having at least two hydroxyl or ether groups, the stated object is achieved in particular in a surprising, in no way foreseeable and, according to the invention, particularly advantageous manner. In contrast to the opinion which can be deduced from the prior art, it is possible, surprisingly and in spite of the presence of a particular water-soluble organic hydrocarbon, to allow the coupled enzymatic reaction system to operate without a solvent-induced loss of activity of one of the enzymes.

Organic hydrocarbons which it has proved preferable to employ are compounds of the general formula (I)

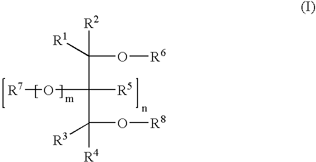

wherein

n is an integer from 0 to 10,

m is 0 or 1,

R¹ to R⁸ independently of one another denote H, (C₁-C₈)-alkyl, (C₂-C₈)-alkoxyalkyl, (C₆-C₈)-aryl, (C₇-C₁₉)-aralkyl,

(C₁-C₈)-alkyl-(C₆-C₁₈ )-aryl, (C₃-C₈)-cycloalkyl,

(C₁-C₈)-alkyl-(C₃-C₈)-cycloalkyl,

(C₃-C₈)-cycloalkyl-(C₁-C₈)-alkyl.

The use of ethylene glycol, DME or glycerol is very particularly preferred in this connection.

The expert is free to choose the amount in which he adds 10 the organic cosolvent to the reaction mixture. The optimum amount can consequently be determined by routine experiments. Addition in an amount of 1-80 is preferred, more preferably 5-60 and very particularly preferably 10-45 vol. %, with respect to the aqueous phase present.

The cofactors which are the most usual and operate most economically under the reaction conditions are preferably used as cofactors. These are, in particular, cofactor NADH or NADPH.

A dehydrogenase is preferably employed as the enzyme for the transformation of the organic compound. In principle, however, the reaction system can also be operated with any other cofactor-dependent oxidoreductase, where the cofactor is consumed by the oxidoreductase and can be regenerated by a second enzymatic system, that is to say the system is a coupled enzymatic system. Further suitable enzymes of this type can be found in the literature (Enzyme Catalysis in Organic Synthesis; Ed.: K. Drauz, H. Waldmann, Vol. I and II, VCH, 1995).

An alcohol dehydrogenase or amino acid dehydrogenase has proved to be an enzyme which it is preferable to employ.

The nature of the regeneration of the cofactor primarily depends on the cofactor employed itself. Various methods of cofactor regeneration can be found in the abovementioned literature. Under the given framework conditions of solvent, enzymes and space/time yield, the expert has a free choice of the regeneration medium. In general, in respect of NAD+ as the cofactor (in oxidation reactions) an NADH oxidase from e.g. Lactobacillus brevis or L. kefir is suitable (DE10140088). In the case of reduction reactions, regeneration of the cofactor NADH by a formate dehydrogenase has furthermore also proved to be very successful.

The invention also relates to a device for the transformation of organic compounds comprising a reaction system according to the invention. This can be e.g. an enzymatic kit.

Devices which are advantageously to be employed are, for example, a stirred tank or cascades of stirred tanks, or membrane reactors, which can be operated both in batch operation and continuously.

In the context of the invention, membrane reactor is understood as meaning any reaction vessel in which the catalyst is enclosed in a reactor, while low molecular weight substances are fed to the reactor or can leave it. The membrane here can be integrated directly into the reaction space or incorporated outside in a separate filtration module, in which the reaction solution flows continuously or intermittently through the filtration module and the retained product is recycled into the reactor. Suitable embodiments are described, inter alia, in WO98/22415 and in Wandrey et al. in Yearbook 1998, Verfahrenstechnik und Chemieingenieurwesen [Process Technology and Chemical Engineering], VDI p. 151 et seq.; Wandrey et al. in Applied Homogeneous Catalysis with Organometallic Compounds, vol. 2, VCH 1996, p. 832 et seq.; Kragl et al., Angew. Chem. 1996, 6, 684 et seq.

The continuous procedure which is possible in this apparatus, in addition to batch and semi-continuous operation, can be carried out here as desired in the cross-flow filtration mode (FIG. 3) or as dead-end filtration (FIG. 2). Both process variants are described in principle in the prior art (Engineering Processes for Bioseparations, ed.: L. R. Weatherley, Heinemann, 1994, 135-165; Wandrey et al., Tetrahedron Asymmetry 1999, 10, 923-928).

The present Application also provides a process for the enzymatic transformation of an organic compound using the reaction system according to the invention. The process is preferably the preparation of an enantiomerically enriched organic compound, preferably an α-amino acid or a chiral alcohol.

The process procedure can be implemented as desired by the expert, with the aid of the reaction system described and the examples described in the following. The conditions which are otherwise known for the enzymatic reaction are set accordingly under the given framework conditions.

One aspect of the invention is also the use of the reaction system according to the invention for the enzymatic transformation of organic compounds or for the diagnosis or analysis of particular organic substances. The enzymatic transformation of an organic compound is preferably carried out in this context with the formation of enantiomerically enriched products.

According to the invention, coupled enzymatic system is understood as meaning that an enzymatic transformation of an organic compound proceeds with the consumption of a cofactor and the cofactor is regenerated in situ by a second enzymatic system. As a result, this leads to a reduction in the use of expensive cofactors.

It is particularly surprising here that in spite of current doctrine the two enzymes employed are not impaired by the presence of the organic medium and it is thus possible to prepare the desired products in very good space/time yields.

As has been shown, for both DME and ethylene glycol, in contrast to most organic solvents (see comparison examples), which lead to rapid deactivation of the FDH employed, outstanding stability properties of the formate dehydrogenase can also still be observed after several days. While, for example, the enzyme activity decreases by 35 and 66% within 24 hours in the presence of acetone and DMSO respectively, 80% of the enzyme activity is still to be recorded even after 5 days in the presence of 10% DME. The results with DME and ethylene glycol are shown on a graph in FIG. 1 and reproduced in table 3. The comparison examples with other organic solvents are also shown in FIG. 1.

The process is carried out both with the wild-type of the formate dehydrogenase from Candida boidinii and with a form of this enzyme modified by genetic engineering (DE19753350). As stated, NADH is preferably employed as the cofactor. For the experimental studies, for example, an ADH from Rhodococcus, preferably Rhodococcus erythropolis, in native or recombinant form can be employed as the ADH component. The enzymes employed can be used for the reaction in any desired purified native or recombinantly prepared form. Their use in the form of the intact whole cells of the host organism is also conceivable. The embodiment in which the two enzyme systems are present in the whole cell catalyst in a state adapted to the optimum reaction is also advantageous in this context (DE10218689).

It is furthermore interesting that the alcohol dehydrogenase also shows a high stability in the presence of organic hydrocarbons of the formula (I). The solvent system according to the invention is thus suitable for carrying out asymmetric biocatalytic reductions. This has been investigated experimentally with the aid of the asymmetric synthesis of 1-p-chlorophenylethan-1-ol or 1-n-butylphenylethan-1-ol starting from p-chloroacetophenone or p-(n-butyl)acetophenone respectively.

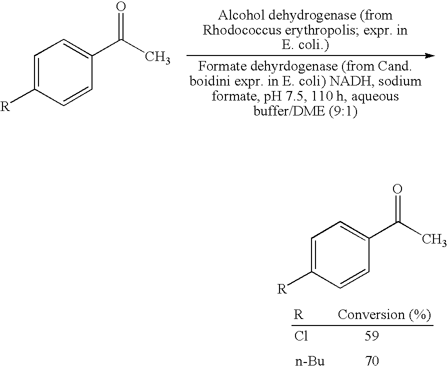

After a reaction time of 110 hours the product was formed with a formation rate of 59% or 70% for p-chloroacetophenone or p-(n-butyl)acetophenone respectively as the substrate.

A main advantage of this process is the simplicity of the process. Thus, it comprises no expensive process steps, and the process can be carried out both in batch reactors and continuously. Likewise, in contrast to earlier processes no special membranes which separate the aqueous medium from the organic medium are required. The surfactant additions required in some processes to date are also omitted in this process. This was not to be seen from the prior art and nevertheless makes the present process extremely advantageous.

As linear or branched (C₁-C₈)-alkyl there are to be regarded methyl, ethyl, n-propyl, isopropyl, n-butyl, isobutyl, sec-butyl, tert-butyl, pentyl, hexyl, heptyl or octyl, including all their bonding isomers. (C₂-C₈)-Alkoxyalkyl means radicals in which the alkyl chain is interrupted by at least one oxygen function, where two oxygen atoms cannot be bonded to one another. The number of carbon atoms indicates the total number of carbon atoms contained in the radical. All the bonding isomers are also included.

A (C₃-C₈)-cycloalkyl radical is understood as meaning cyclopropyl, cyclobutyl, cyclopentyl, cyclohexyl or cycloheptyl radicals etc. A cycloalkyl radical substituted by heteroatoms is preferably e.g. 1-, 2-, 3-, 4-piperidyl, 1-, 2-, 3-pyrrolidinyl, 2-, 3-tetrahydrofuryl, 2-, 3-, 4-morpholinyl.

A (C₃-C₈)-cycloalkyl-(C₁-C₈)-alkyl radical designates a cycloalkyl radical as described above which is bonded to the molecule via an alkyl radical as mentioned above.

A (C₆-C₁₈)-aryl radical is understood as meaning an aromatic radical having 6 to 18 C atoms. This includes, in particular, radicals such as phenyl, naphthyl, anthryl, phenanthryl and biphenyl radicals.

A (C₇-C₁₉)-aralkyl radical is a (C₆-C₁₈)-aryl radical bonded to the molecule via a (C₁-C₈)-alkyl radical.

Enantiomerically enriched describes the fact that one optical antipode is present in a mixture with its other to >50%.

The structures shown relate to all the possible diastereomers and, in respect of a diastereomer, to the two possible enantiomers of the compound in question which fall under this.

A homogeneous aqueous solvent system is understood according to the invention as meaning that the hydrocarbon employed forms a homogeneous solution with the aqueous phase, and consequently only one liquid phase is thus present.

The process according to the invention is illustrated by the example described below.

### DESCRIPTION OF THE DRAWINGS

FIG. 2 shows a membrane reactor with dead-end filtration. The substrate 1 is transferred via a pump 2 into the reactor space 3, which contains a membrane 5. In the reactor space, which is operated with a stirrer, are, in addition to the solvent, the catalyst 4, the product 6 and unreacted substrate 1. Low molecular weight 6 is chiefly filtered off via the membrane 5.

FIG. 3 shows a membrane reactor with cross-flow filtration. The substrate 7 is transferred here via the pump 8 into the stirred reactor space, in which are also solvent, catalyst 9 and product 14. A solvent flow which leads via a heat exchanger 12, which may be present, into the cross-flow filtration cell 15 is established via the pump 16. The low molecular weight product 14 is separated off here via the membrane 13. High molecular weight catalyst 9 is then passed back with the solvent flow, if appropriate via a heat exchanger 12 again, if appropriate via the valve 11, into the reactor 10.

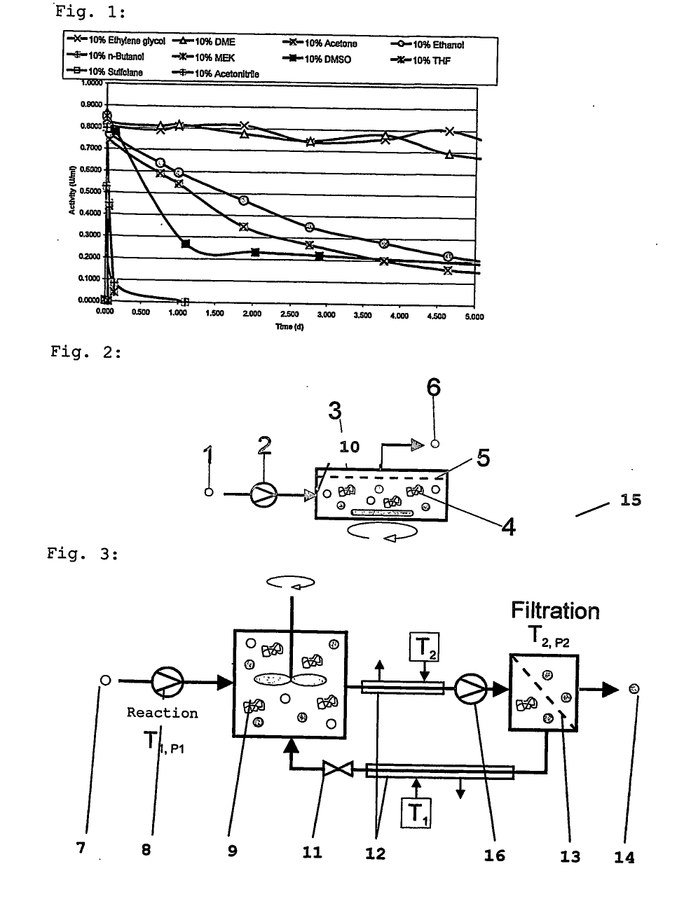

### EXPERIMENTAL PART

### EXAMPLE 1

### Comparison Examples of FDH Activities

2.72 g (0.8 mol/l) sodium formate and 1.14 g (0.1 mol/l) di-potassium hydrogen phosphate trihydrate are weighed out and are dissolved in 40 ml of completely demineralized H₂O. The pH of the solution is adjusted to 8.2 with ammonia solution (25%) and formic acid (100%) or appropriate dilutions. The solution is then transferred to a 50 ml volumetric flask and topped up with completely demineralized H₂O. Separately to this, 71.7 mg (4 mmol/l) NAD⁺ trihydrate are weighed out and dissolved in approx. 20 ml of completely demineralized H₂O. The pH of the solution is adjusted to 8.2 with ammonia solution (25%) and formic acid (100%) or appropriate dilutions. The solution is then transferred to a 25 ml volumetric flask and topped up with completely demineralized H₂O. In each case 500 μl of the substrate solution and of the NADH solution are then mixed in the 1 cm cell used for the measurement. After addition of 10 μl of the enzyme solution, a 10% solution of an organic solvent (see table) in water being employed as the solvent, the mixture is shaken briefly, the cell is placed in the photometer and recording of the data is started. The enzyme solution is added only directly before the start of the measurement. The activities of the enzymes are determined after certain intervals of time by photometric detection of the reaction of NAD⁺ to give NADH. The photometric measurement was carried out at a temperature of 30° C. and a wavelength of 340 nm with a measurement time of 15 min. The results are shown in the following in table 1 and table 2.

TABLE 1
Enzyme activity of the FDH in U/ml as
a function of the solvent and time

| | Butanol | MEK | DMSO | THF | Sulfolane | Acetonitrile |
|---|---|---|---|---|---|---|
| Time | Activity | Activity | Activity | Activity | Activity | Activity |
| [d] | [U/ml] | [U/ml] | [U/ml] | [U/ml] | [U/ml] | [U/ml] |
| 0.000 | 0.5262 | 0.0058 | 0.7965 | 0.8492 | 0.0028 | 0.7961 |
| 0.042 | 0.0006 | 0.0011 | 0.7880 | 0.4357 | 0.0003 | 0.4494 |
| 0.125 | | | 0.7794 | 0.0414 | | 0.0840 |
| 1.097 | | | 0.2669 | | | 0.0008 |
| 2.035 | | | 0.2331 | | | |
| 2.896 | | | 0.2201 | | | |
| 5.927 | | | 0.1763 | | | |
| 7.885 | | | 0.1404 | | | |
| 9.948 | | | 0.1205 | | | |
| 13.073 | | | 0.0915 | | | |
| 14.892 | | | 0.0717 | | | |
| 16.875 | | | 0.0540 | | | |
| 19.938 | | | 0.0355 | | | |

TABLE 2
Enzyme activity of the FDH in U/ml as
a function of the solvent and time

| | Acetone | Ethanol |
|---|---|---|
| Time | Activity | Activity |
| [d] | [U/ml] | [U/ml] |
| 0.000 | 0.8355 | 0.8491 |
| 0.042 | 0.7402 | 0.7689 |
| 0.750 | 0.5893 | 0.6367 |
| 1.000 | 0.5426 | 0.5933 |
| 1.875 | 0.3484 | 0.4687 |
| 2.760 | 0.2691 | 0.3510 |
| 3.781 | 0.2004 | 0.2814 |
| 4.646 | 0.1614 | 0.2240 |
| 5.875 | 0.1325 | 0.1736 |
| 6.778 | 0.0987 | 0.1486 |
| 7.792 | 0.0794 | 0.1277 |
| 8.729 | 0.0610 | 0.0998 |
| 11.750 | 0.0333 | 0.0536 |
| 13.726 | | 0.0421 |

### EXAMPLE 2

### Measurement of the FDH Activities

The activity was determined in accordance with the instructions in example 1, DME and ethylene glycol being used as the organic solvent component. The results are shown in the following in table 3.

TABLE 3
Enzyme activity of the FDH in U/ml as
a function of the solvent and time

| Time | Ethylene glycol | DME |
|---|---|---|
| [d] | Activity [U/ml] | Activity [U/ml] |
| 0.000 | 0.8325 | 0.8668 |
| 0.042 | 0.8053 | 0.8210 |
| 0.750 | 0.7891 | 0.8095 |
| 1.000 | 0.8070 | 0.8160 |
| 1.875 | 0.8146 | 0.7767 |
| 2.760 | 0.7415 | 0.7471 |
| 3.781 | 0.7588 | 0.7789 |
| 4.646 | 0.8011 | 0.6943 |
| 5.875 | 0.6585 | 0.6535 |
| 6.778 | 0.5697 | 0.6495 |
| 7.792 | 0.4479 | 0.6391 |
| 8.729 | 0.2463 | 0.5565 |
| 11.750 | 0.0066 | 0.4814 |
| 13.726 | | 0.4610 |
| 15.969 | | 0.4272 |
| 18.819 | | 0.3613 |
| 20.948 | | 0.3249 |
| 22.750 | | 0.2976 |
| 25.781 | | 0.2703 |
| 27.740 | | 0.2374 |
| 29.802 | | 0.2216 |
| 32.927 | | 0.1998 |
| 34.747 | | 0.1883 |
| 36.729 | | 0.1658 |
| 39.792 | | 0.158634 |
| 42.830 | | 0.143468 |
| 60.708 | | 0.0943626 |
| 75.781 | | 0.0470678 |

### EXAMPLE 3

A reaction mixture, comprising 25 mM p-chloroacetophenone, as well as 0.1 mM NAD⁺, and 75 mM sodium formate at enzyme concentrations of 0.1 U/mi S-ADH and 0.2 U/ml FDH (DM), is stirred at a reaction temperature of 30° C. over a period of 110 hours in a solvent system comprising 90 vol. % 100 mM phosphate buffer with a pH of 7.5 and 10 vol. % DME. The organic components are then extracted with methylene chloride, the aqueous phase is discarded and the organic phase is dried over sodium sulfate. The filtrate which results after filtration is freed from the readily volatile constituents in vacuo and the resulting oil is investigated in respect of the formation rate by analysis via ¹H nuclear magnetic resonance spectroscopy. A formation rate of 59% was determined.

### EXAMPLE 4

A reaction mixture, comprising 25 mM p-(n-butyl)acetophenone, as well as 0.1 mM NAD⁺, and 75 mM sodium formate at enzyme concentrations of 0.2 U/ml S-ADH and 0.4 U/ml FDH (DM), is stirred at a reaction temperature of 30° C. over a period of 110 hours in a solvent system comprising 90 vol. % 100 mM phosphate buffer with a pH of 7.5 and 10 vol. % DME. The organic components are then extracted with methylene chloride, the aqueous phase is discarded and the organic phase is dried over sodium sulfate. The filtrate which results after filtration is freed from the readily volatile constituents in vacuo and the resulting oil is investigated in respect of the formation rate by analysis via ¹H nuclear magnetic resonance spectroscopy. A formation rate of 70% was determined.

### EXAMPLE 5

### Reaction with a 20% (v/v) ethylene glycol Content

A reaction mixture, comprising 1.3 mmol p-chloroacetophenone (208.3 mg; 13 mM), as well as 0.24 mmol NADH (169.4 mg; 2.4 mM), and 6.2 mmol sodium formate (62 mM; 421.7 mg; 4.8 equivalents, based on the ketone) at enzyme amounts of 24 U of an (S)-ADH from R. erythropolis (expr. in E. coli) and 24 U of a formate dehydrogenase from Candida boidinii (double mutants: C23S, C262A; expr. in E. coli), is stirred at a reaction temperature of 30° C. over a period of 21 hours in a solvent system comprising 80 vol. % 50 mM phosphate buffer with a pH of 7.0 and 20 vol. % ethylene glycol. Samples are taken during this period of time and the particular conversion is determined via PLC. After 21 hours, complete conversion of the ketone was found. The organic components are then extracted with 4×100 ml methyl tert-butyl ether, the aqueous phase is discarded and the organic phase is dried over sodium sulfate. The filtrate which results after filtration is freed from the readily volatile constituents in vacuo and the resulting residue, after further addition of MTBE and separation of the two phases formed, is investigated in respect of the formation rate by analysis via ¹H nuclear magnetic resonance spectroscopy. A formation rate of >99% was determined.

### EXAMPLE 6

### Reaction with a 40% (v/v) ethylene glycol Content

A reaction mixture, comprising 2.63 mmol p-chloroacetophenone (407.3 mg; 26.3 mM), as well as 0.52 mmol NADH (372.1 mg; 5.2 mM), and 14.4 mmol sodium formate (144 mM; 979.3 mg; 5 equivalents, based on the ketone) at enzyme amounts of 52.4 U of an (S)-ADH from R. erythropolis (expr. in E. coli) and 52.4 U of a formate dehydrogenase from Candida boidinii (double mutants: C23S, C262A; expr. in E. coli), is stirred at a reaction temperature of 30° C. over a period of 21 hours in a solvent system comprising 60 vol. % 50 mM phosphate buffer with a pH of 7.0 and 40 vol. % ethylene glycol. Samples are taken during this period of time and the particular conversion is determined via PLC. After 21 hours, complete conversion of the ketone was found. The organic components are then extracted with 2×100 ml methyl tert-butyl ether, the aqueous phase is discarded and the organic phase is dried over sodium sulfate. The filtrate which results after filtration is freed from the readily volatile constituents in vacuo and the resulting residue, after further addition of MTBE and separation of the two phases formed, is investigated in respect of the formation rate by analysis via ¹H nuclear magnetic resonance spectroscopy. A formation rate of >99% was determined.

1. A coupled enzymatic reaction system comprising a cofactor-dependent enzymatic transformation of an organic compound and an enzymatic regeneration of the cofactor, wherein the reaction system operates in a homogeneous aqueous solvent system comprising an organic hydrocarbon with at least two hydroxyl or ether groups.

2. The reaction system according to claim 1, wherein
the organic hydrocarbon employed has the structure according to the general formula (I) 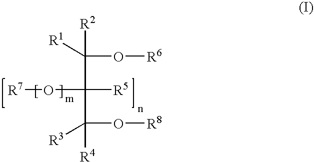
wherein
n is an integer from 0 to 10, m is 0 or 1
R¹ to R⁸, independently of one another, denote H, (C₁-C₈)-alkyl, (C₂-C₈)-alkoxyalkyl, (C₆-C₁₈)-aryl, (C₇-C₁₉)-aralkyl, (C₁-C₈)-alkyl-(C₆-C₁₈)-aryl, (C₃-C₈)-cycloalkyl, (C₁-C₈)-alkyl-(C₃-C₈)-cycloalkyl, or (C₃-C₈)-cycloalkyl-(C₁-C₈)-alkyl.

3. The reaction system according to claim 1, wherein
ethylene glycol, DME or glycerol are used as the organic hydrocarbon.

4. The reaction system according to claim 1,
wherein
the organic hydrocarbon is present in an amount of
1-80 vol %, with respect to the aqueous phase.

5. The reaction system according to claim 1,
wherein
NADH or NADPH is employed as the cofactor.

6. The reaction system according to claim 1,
wherein
a dehydrogenase is employed as the enzyme for the transformation of the organic compound.

7. The reaction system according to claim 6,
wherein
an alcohol dehydrogenase or an amino acid dehydrogenase is employed.

8. The reaction system according to claim 1,
wherein
the regeneration of the cofactor takes place by means of a formate dehydrogenase.

9. A device for the transformation of organic compounds comprising a reaction system according to claim 1.

10. A process for the enzymatic transformation of organic compounds comprising enzymatically transforming an organic compound with the reaction system according to claim 1.

11. A process for diagnosis or analysis of organic compounds comprising diagnosing or analyzing organic compounds with the reaction system of claim 1.

12. The process according to claim 10, wherein enantiomerically enriched organic compounds are prepared.
